(19) United States
(12) Patent Application Publication
(10) Pub. No.: US 2021/0161572 A1
(43) Pub. Date: Jun. 3, 2021

(54) FLOWABLE BIOACTIVE BONE VOID FILLER

(71) Applicant: PROSIDYAN, INC., New Providence, NJ (US)

(72) Inventors: Charanpreet S. Bagga, Basking Ridge, NJ (US); Shrikar P. Bondre, East Windsor, NJ (US)

(21) Appl. No.: 17/168,589

(22) Filed: Feb. 5, 2021

Related U.S. Application Data
(63) Continuation-in-part of application No. 16/294,138, filed on May 24, 2019.
(60) Provisional application No. 62/639,099, filed on Mar. 6, 2018.
(60) Provisional application No. 62/970,835, filed on Feb. 6, 2020.

(51) Int. Cl. A61B 17/88; A61L 27/12; A61L 27/20; A61L 27/26

(52) U.S. Cl. CPC A61B 17/8805; A61L 27/12; A61L 27/20; A61L 2300/802; A61L 2400/06; A61L 2430/02; A61L 27/26

(57) ABSTRACT

A flowable, bioactive bone void filler is provided. This bone void filler may be a settable, hardening material having sufficient compression strength for use in bone repair techniques. The cement may be a calcium phosphate cement having incorporated therein bioactive glass, and can be used as a bone graft substitute or bone void filler for any number of applications in spine surgery and orthopedic surgery, such as for example, subchondral bone repair.

### CROSS-REFERENCE TO RELATED APPLICATIONS

This application is a Continuation-In-Part of U.S. patent application Ser. No. 16/294,138 filed Mar. 6, 2019, which claims benefit of U.S. Provisional No. 62/639,099 filed Mar. 6, 2018. This application also claims benefit of U.S. Provisional No. 62/970,835 filed Feb. 6, 2020. The contents of all of these are herein incorporated by reference in their entirety.

### TECHNICAL FIELD

The present disclosure relates generally to materials for treating bone fractures, voids, lesions or other bone defects. More specifically, the present disclosure provides a bone cement or bone void filler for use in stabilizing bone fractures, voids, lesions or other defects.

### BACKGROUND

One of the most widely accepted medical procedures to treat fractures, voids, lesions, bruises or other defects of the bone that result in its weakening or instability is to stabilize the damaged bone region with a hardening material, such as a bone cement or bone void filler. The cement or filler may be inserted into an interior cavity of the bone, or placed on or over the damaged area, and act to stabilize the weakened, damaged or diseased bone region, enhancing strength and reducing susceptibility to collapse. These bone cements and bone void fillers may also serve as a bone graft substitute. For this reason, it is beneficial to have bone cements and bone void fillers that also include additional bone growth or bone enhancing properties. For instance, it would be desirable to provide a material that is suitable for use as a bone cement or bone void filler to provide the necessary structural stabilization to strengthen weakened bone, but is also osteostimulative and bioactive so as to biologically treat the bone as well.

As an example, calcium phosphate based materials are commonly used nowadays as a bone graft substitute or bone void filler for a number of applications in spine surgery and orthopedic surgery. One such orthopedic application is subchondral bone repair, a minimally invasive procedure used to relieve the patient of the pain and discomfort caused by a bone marrow lesion in the knee, a complex environment that is made up of bone, cartilage, ligaments, muscle and fluid. See, Subchondroplasty: A New Option for Arthritis. Mathew Pombo, MD Assistant Professor Emory University, Department of Sports Medicine, Jan. 14, 2016. Emoryhealthcare.org/ortho; and Subchondral Bone Treatment. Geoffrey D. Abrams, MD; Joshua D. Harris, MD; and Brian J. Cole, MD, MBA, Chapter 12 Biologic Knee Reconstruction: A Surgeon's Guide (pp 83-89). A bone marrow lesion is a microfracture or swelling in the bone right below the knee joint that is generally caused by osteoarthritis. Calcium phosphate (CaP) bone void fillers have been developed with the goal to improve the integrity of damaged bone. In subchondral bone repair, the CaP paste is prepared and injected directly into the damaged area of the knee. See, Subchondroplasty: Filling the Void in Your Knee. STARS Physical Therapy, Saint Alphonsus. CaP fillers are primarily osteoconductive materials. However, there is room to improve upon these calcium phosphate materials, including the addition of biologically enhancing components. Additionally, it is desirable to provide these biologically improved materials in a flowable, injectable form for ease of application.

### SUMMARY

The present disclosure provides an osteostimulative, bioactive and flowable bone void filler or bone cement. The cement may be a calcium phosphate cement having incorporated therein bioactive glass, and can be used as a bone graft substitute or bone void filler for any number of applications in spine surgery and orthopedic surgery, such as in one particular application, for subchondral bone repair. The bioactive glass component may comprise particles having relatively small diameters (i.e., less than about 10 microns) to provide greater interdigitation with the trabeculae of the cancellous bone, without compromising compressive strength.

In one exemplary embodiment of the present disclosure, a bone void filler for treating a bone defect is provided. The bone void filler may comprise a calcium phosphate material having therein a bioactive glass component, the bone void filler being osteostimulative, bioactive and flowable for injection through a syringe. This bone void filler may be a settable, hardening material having sufficient compression strength for use in bone repair techniques.

According to one aspect of the embodiment, the calcium phosphate material may comprise 15 to 40% by wt. beta tricalcium phosphate. The bone void filler may further include 15 to 30% by wt. monocalcium phosphate monohydrate, 5 to 15% by wt. hydroxyapatite, 5 to 7% by wt carboxy methyl cellulose, and 5% to 35% surfactant such as copolymers of polypropylene polyethylene glycol.

According to another aspect of the embodiment, the bioactive glass component may be in the range of 5 to 25% by wt. bioactive glass and have an average diameter in the range of 50 to 200 microns. The bioactive glass component may comprise 45S5 bioactive glass (45 wt % SiO2, 24.5 wt % CaO, 24.5 wt % Na2O and 6.0 wt % P2O5), boron bioactive glass (20 wt % CaO, 6 wt % Na2O, 4 wt % P2O5, 51.6 wt % B2O3, 12 wt % K2O, 5 wt % MgO, 0.4 wt % CuO, 1 wt % ZnO), or S53P4 (53 wt % SiO2, 23 wt % Na2O, 20 wt % CaO and 4 wt % P2O5).

After hardening, the bone void filler may have a minimum compressive strength of 1 mPa. Accordingly, the bone void filler may be suitable for use in treating a bone defect where the defect is a bone marrow lesion, and the filler is injected in a subchondral bone defect.

In another exemplary embodiment of the present disclosure, a kit may be provided for making a bone void filler or bone cement for treating a bone defect. The kit may comprise: (A) dry components of calcium phosphate and bioactive glass, and (B) a liquid component comprising saline, citric acid, or sodium hydroxide solution. The dry material may comprise 5 to 25% by wt. bioactive glass powder, 15 to 40% by wt. beta tricalcium phosphate powder, 15 to 30% by wt. monocalcium phosphate monohydrate, 5 to 15% by wt. hydroxyapatite, 5 to 7% by wt. carboxy methyl cellulose, and 5% to 35% surfactant such as copolymers of polypropylene polyethylene glycol. The liquid component may comprise 0.5 M solution in a ratio of 2.2 gram dry material/cc of liquid.

In one embodiment, the bioactive glass powder may have an average diameter in the range of 75 to 200 microns. In another embodiment, the bioactive glass powder may have an average diameter in the range of about 2 to 25 microns. In yet another embodiment, the bioactive glass powder may have an average diameter of less than 10 microns.

In some embodiments, the kit may include a delivery system that may be configured to hydrate, mix and deliver the material. The delivery system may include syringes for containing the dry and liquid components. For instance, the delivery system may include a first syringe for containing the dry components and a second syringe for containing the liquid component. The second syringe may be configured to attach to the first syringe with a connector component of the delivery system. An exemplary delivery system useful with the present material is the Medmix P-System by Medmix Systems AG of Rotkruez, Switzerland.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a photograph of a two (2) component system for preparing the bone void filler or bone cement of the present disclosure.

FIG. 2A is an exploded view of an exemplary delivery system for the bone void filler or bone cement of FIG. 1.

FIG. 2B is a perspective view of the delivery system of FIG. 2A assembled with the dry components of the component system of FIG. 1.

FIGS. 3A-3F are photographs showing an exemplary method of preparing the bone void filler with the delivery system of the present disclosure, in which:

FIG. 3A shows an exploded view of the delivery system of FIG. 1 in which a main syringe containing dry components is attached to a paddle plunger, without the push plunger and cap attached.

FIG. 3B shows the main syringe containing dry components of FIG. 3A attached to a second syringe containing a liquid component for mixing with the dry components.

FIG. 3C shows the main syringe containing the mixed dry and liquid components of FIG. 3B with a Luer cap attached to the syringe cap.

FIG. 3D shows a step of attaching the push plunger and paddle plunger together and onto the main syringe of FIG. 3C.

FIG. 3E shows the push plunger and paddle plunger of FIG. 3D assembled together and attached to the main syringe.

FIG. 3F shows a step of delivering the bone void material within the main syringe by depressing the assembled push plunger and paddle plunger of FIG. 3E.

FIG. 4 is a photograph of a paste formed of calcium phosphate bone cement infused with bioactive glass particles.

FIGS. 5A and 5B are photographs of solid bone grafts formed of the paste of FIG. 4.

FIG. 6 is a graphical representation of compression strength of Formulations 1, 2 and 3 of the paste of FIG. 4 over time.

### DESCRIPTION OF THE EMBODIMENTS

The present disclosure provides an osteostimulative, bioactive and flowable bone void filler or bone cement. This bone void filler or bone cement may be a settable, hardening material having sufficient compression strength for use in bone repair techniques. The cement may be a calcium phosphate cement having incorporated therein bioactive glass, and can be used as a bone graft substitute or bone void filler for any number of applications in spine surgery and orthopedic surgery, such as in one particular application, for subchondral bone repair.

Various calcium phosphates are contemplated and include, for example, tricalcium phosphate, β-tricalcium phosphate (β-TCP), α-tricalcium phosphate (α-TCP), monocalcium phosphate monohydrate, and apatites such as hydroxyapatite. However, for the sake of brevity, “calcium phosphate” includes any calcium salt known to those skilled in the art. According to one aspect of the embodiment, the bone void filler or cement is a multicomponent, hydrolysable material comprising different types of calcium phosphates. In one embodiment, the calcium phosphate(s) are in powder form. The calcium phosphates in the formulation may be in the range of 30% to 99% by wt., 50% to 98% by wt. or 75% to 95% by wt. of the dry components. For example, the dry formulation may comprise 15% to 40% by wt β-tricalcium phosphate, 15% to 30% by wt monocalcium phosphate monohydrate and 5% to 15% by wt hydroxyapatite.

The multicomponent, hydrolysable material may further comprise carboxymethyl cellulose (CMC), poloxamer or other cellulosics. In one aspect, the cellulose material comprises 5% by wt or less of the dry material.

Bioactive glass is a category of glass having bioactive properties, the use of which has an established history of bone bonding that occurs as a result of a rapid sequence of reactions on its surface when implanted into living tissues. When hydrated, a layer of silica gel forms on the surface of the bioactive glass. The adhesion of amorphous calcium, phosphate, and carbonate ions to the silica surface leads to an eventual crystallization of a bone-like hydroxyapatite (HA) in as early as 24 hours. Bone-forming cells migrate and colonize the surface of the bioactive glass and promote the production of a new bone like matrix. The addition of an osteostimulative material such as bioactive glass will help the general healing response.

The dry multicomponent calcium phosphate materials may further comprise a bioactive glass such as 45S5 or a borate glass such as S53P4 for example, although it is understood that other bioactive glasses may also be used as well. In one aspect, the bioactive glass is in powder form. The bioactive glass particles may range in size from about 2 μm to 200 μm, 75 μm to 125 μm, 50 μm to 100 μm, 60 μm to 90 μm, and less than about 10 μm, such as for example, 2 μm to 25 μm. In one embodiment, the bioactive glass particles have a diameter of less than about 10 μm.

In one aspect, each component in the dry formulation is less than 200 μm.

In another aspect, the liquid to powder ratio is in the range of about 0.25 to 0.4. In some embodiments, the liquid to powder ratio is about 0.3 to 0.35.

The dry components of the bone cement or bone void filler may be hydrated with an aqueous solution. In one aspect, the bone cement or bone void filler is hydrated with a citric acid solution. The citric acid solution is typically 0.45M to 0.55M, preferably 0.5M. The ratio of dry to liquid components may be 2.0 g to 2.5 g dry per cc of liquid. In one embodiment, the ratio is 2.2 g dry/cc liquid.

### WORKING EXAMPLES

The following describes exemplary working examples of the bioactive glass infused bone void filler.

### Example 1: Preparation of an Exemplary Bone Void Filler

In one exemplary embodiment, the bone cement or bone void filler material can be prepared from a two (2) component system 10 that consists of: (A) dry components of calcium phosphate and bioactive glass, and (B) a wet solution such as saline, citric acid, or sodium hydroxide solution. As an example, the dry material (a) can comprise from 5 to 25% by wt. bioactive glass powder (50 to 200 microns), 15 to 40% by wt. beta tricalcium phosphate (TCP) powder, 15 to 30% by wt. monocalcium phosphate monohydrate (MCPM), 5 to 15% by wt. hydroxyapatite (HA), and 5 to 7% by wt. carboxy methyl cellulose (CMC). Additional, 5% to 35% surfactant such as copolymers of polypropylene polyethylene glycol may also be added.

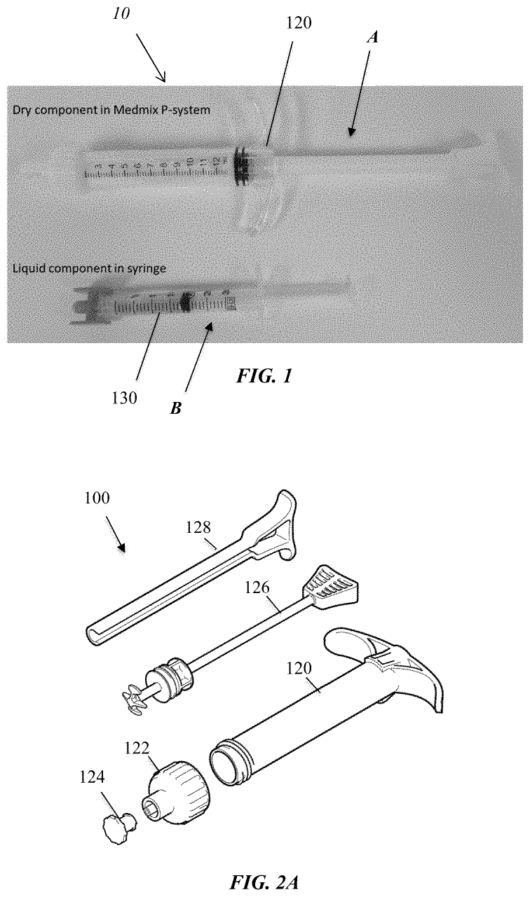

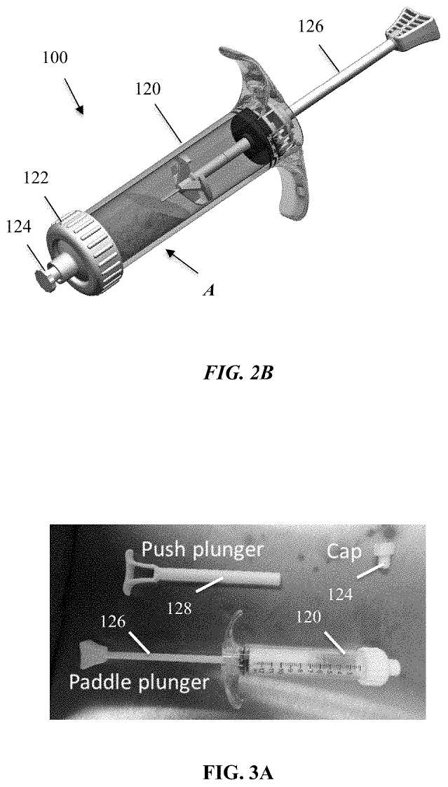

The premixed dry components may be loaded into a syringe 120, while the liquid component may be loaded into a separate syringe 130, as shown in FIG. 1. The syringes 120, 130 may be part of a bone cement delivery system 100, such as for example, the Medmix P-System by Medmix Systems AG of Rotkruez, Switzerland, as shown in FIGS. 2A and 2B. The wet, or liquid, component (b) may consist of 0.5 M solution of citric acid in a ratio of 2.2 gram dry material/cc of liquid.

The delivery system 100 may include a primary, or main, syringe 120 that can hold the dry components (A) of the bone cement or bone void filler system 10. The syringe 120 may attach to a syringe cap 122, which may connect to, and be closed off with, a Luer-cap 124. The syringe 120 may be configured to receive a combination mixing device or paddle plunger 126 and push plunger 128. The push plunger may be configured as a snap-on component (i.e., semi-circular elongate shell or C-sectional shaft) to the paddle plunger 126 and when assembled together, acts as a unitary cylindrical plunger. An assembled delivery system 100 is shown in FIG. 2B for reference. The main syringe 120 of the delivery system 100 contains the dry components (A), similar to FIG. 1.

The bone void filler/bone cement of the present disclosure may be prepared in the following steps:

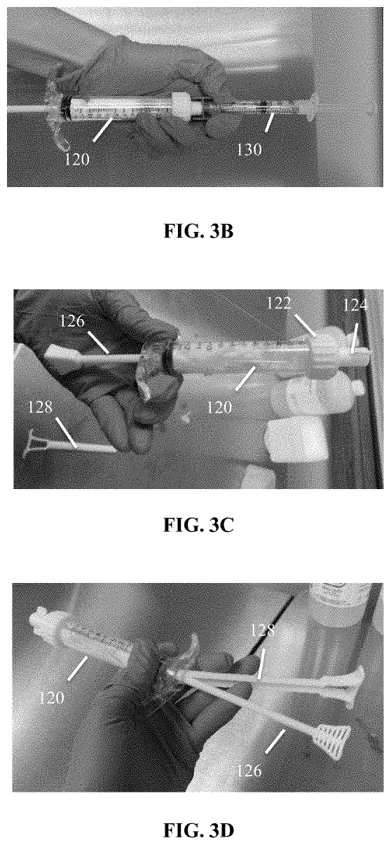

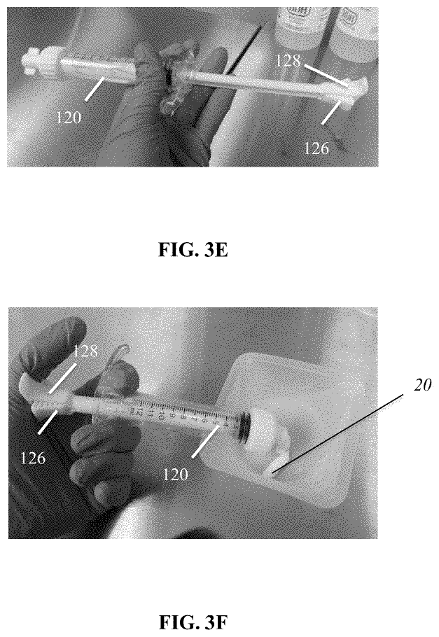

Step 1: With the combination push plunger 128 and paddle plunger 126, pull the dry components (A) (i.e., powder) towards the bottom of the syringe 120, then remove the combination mixing device 126 and push plunger 128 (configured to nest together as a single cylindrical component) and the syringe cap 122 (see FIG. 3A).

Step 2: Using the Luer connector on the Luer-cap 124, attach the second syringe 130 containing the liquid component (B) to the first syringe 120 containing the dry components (A) and then transfer the liquid component (B) into the first syringe 120, as shown in FIG. 3B. It may be desirable to aspirate the liquid component from the syringe 130 one or more times by pulling on the plunger 128. After the liquid component has been transferred, reconnect the syringe cap 122 to the first syringe 120.

Step 3: Separate the empty second syringe 130 from the first syringe 120 by twisting off, then close the first syringe 120 by fixing the Luer cap 124 on the syringe cap 122 on the first syringe 120 (see FIG. 3C).

Step 4: Remove the push plunger 128 from the first syringe 120 to leave behind the mixing device or paddle plunger 126 that was nested within the plunger 128 (see FIG. 3D). Next, mix the liquid component (B) into the dry components (A) by moving the mixing device 126 (i.e., paddle plunger) up and down and simultaneously rotating, for approximately 30 seconds, until all the powder is hydrated and forms a paste, ensuring the mixing is complete at both ends of the syringe 120.

Step 5: Reattach the push plunger 128 onto the mixing device 126 by pulling back the mixing device or paddle plunger 126 completely, aligning the push plunger 128 to the syringe opening, then snapping the push plunger 128 onto the mixing device 126 to form a unitary cylindrical instrument once again (see FIGS. 3D and 3E).

Step 6: Remove the Luer-cap 124 and vent air slowly by compressing the formed paste 20 by pushing on the plungers 126, 128 until all air is removed (see FIG. 3F).

Once the paste 20 has been compressed, the syringe cap 122 can be removed and a syringe accessory such as a syringe needle can be attached in its place to extrude the paste 20. It is understood that there could be some residual paste 20 in the syringe 120. It should be noted that the paste 20 formed can be injected through an 8G cannula, for example.

According to one aspect of the disclosure, the formulation of the paste 20 provides the ability to be injected into a wet or dry environment. The paste 20 has a working time of about 2 to 5 minutes after injection. The setting time is about 5 minutes, while the total hardening time is about 10 minutes. After hardening, the material has a minimum compressive strength of 1 Mpa. After setting, the material forms an apatite that is similar to bone. The material after hardening can also be drilled if desired.

Of course, it is understood that in some applications where the bone is very dense, such as for the treatment of bone marrow lesions of a shoulder joint, as an example, the present material does not need to be settable. In addition to being non-settable, in other embodiments, the bone void filler material may be in the form of a putty. Further, while the bioactive glass component is described in the example above as being in powder form, it is well contemplated that bioactive glass fibers and fibrous mixtures (e.g., fibers plus granules) may be utilized as well. Since the benefits of bioactive glass are well accepted, one can envision a bone void filler material that maximizes the concentration of the bioactive glass, such that it is greater than 25% by wt. and in some cases can be 50 to 85 by wt. or greater. In some embodiments, the bone void filler material may be mostly bioactive glass, whether in powder (granular) or fiber form, or some combination thereof, and having little or no calcium phosphate. For example, a boron-based bioactive glass component with a polymer component such as PEG (polyethylene glycol) may be suitable for use as a bone cement or bone void filler.

In addition, it is contemplated that various syringes may be utilized with the present material. For example, the materials of the present disclosure may be used with a straight syringe, a threaded spindle drive (for mechanical leverage), a reduced diameter syringe, a set of reduced diameter syringes, and a pneumatic, hydraulic or electrically power injection mechanism. For use with power driven mechanisms, the appropriate aliquot of material injections may be calculated and utilized (e.g., 0.1 cc increments) to avoid damage.

Further, while various injection systems may be used for delivering the present material, it is understood that one may elect to apply the paste material 20 in other ways as well. The paste 20 may be formed and then spread onto the treatment site, or applied through any variety of needles, cannulas or other delivery tubes, either with or without additional force such as with suction or vacuum force, pressure, etc.

Overall, the bone void material of the present disclosure is intended to provide a compression resistant scaffold that provides structural integrity to the defect site. The calcium phosphate provides the osteocondutive property. The bioactive glass is intended to provide the osteoconductive and the osteostimulative properties. The surface reactions from the bioactive glass will lead to an eventual crystallization of a bone-like hydroxyapatite (HA) in as early as 24 hours that results in improved osseointegration. Bone-forming cells migrate and colonize the surface of the bioactive glass and promote the production of new bone. In addition, the bioactive glass also helps with the setting of the cement and to provide improved working time for the material. Suitable bioactive glasses can include 4555 bioactive glass (45 wt % SiO₂, 24.5 wt % CaO, 24.5 wt % Na₂O and 6.0 wt % P₂O₅), boron bioactive glass (20 wt % CaO, 6 wt % Na₂O, 4 wt % P₂O₅, 51.6 wt % B₂O₃, 12 wt % K₂O, 5 wt % MgO, 0.4 wt % CuO, 1 wt % ZnO), or other suitable bioactive glasses such as S53P4 (53 wt % SiO₂, 23 wt % Na₂O, 20 wt % CaO and 4 wt % P₂O₅).

### Example 2: Comparative Study to Evaluate Compression Strength

Objective

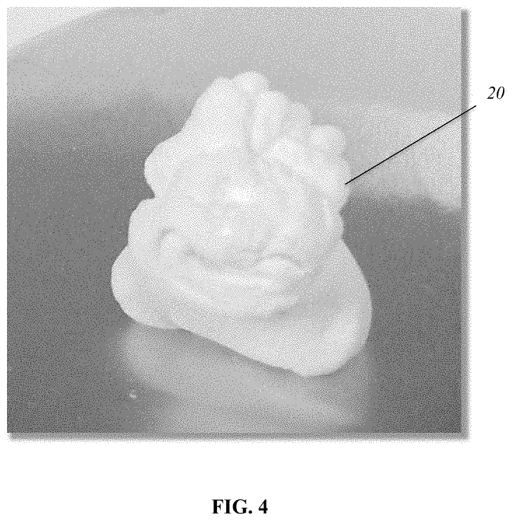

The objective of this study was to evaluate the compressive strength of three calcium phosphate cement formulations containing various percent weight and sizes of bioactive glass. (See FIG. 4 for a photograph of an exemplary calcium phosphate cement formulation as a paste 20).

Background

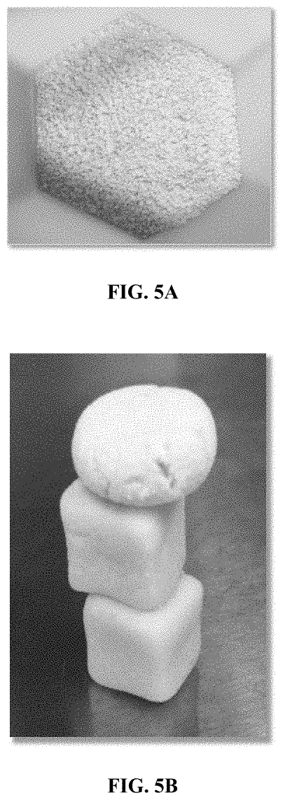

When large cavities or fractures occur in the bone, often times a bone graft is necessary. Calcium Phosphate Cement (CPC) is a preferable bone graft due to its biocompatibility and ability to be easily injected and molded into bone voids [1]. It is also osteoconductive and resorbable, therefore providing a scaffold for osteoblasts to land and promote the body's bone remodeling process. CPC is a combination of one or more different types of calcium phosphates [2]. For this study, the combination of CPC and bioactive glass (BG) were evaluated. Calcium phosphate cements can benefit from the addition of BG 45S5 due to its ability to promote the proliferation, differentiation, mineralization and attachment of osteoblastic cells [3, 4]. A typical CPC cement consists of two components, a powder (P) and a liquid (L). When the two components are combined, the components undergo a reaction that turns the mixture into a solid. (See FIGS. 5A and 5B). Calcium phosphate cements are often used in subchondroplasty procedures, thus the compressive strength of cancellous bone (5-10 MPa) is a relevant benchmark for the material's compressive strength performance [5]. For this study, the compressive strength of three different formulations of CPC combined with and without bioactive glass were evaluated.

Methods

Compression testing was performed in accordance with ASTM D695 Compressive Strength standard. The CPC formulations were cured at 37° C. in a stainless-steel compression mold to provide five columns of 6 mm (diameter) by 12 mm. The formulations differ in composition and percent weight of each material. All formulations contained calcium phosphate with different percent weight of bioactive glass. Formulation 1 was composed of bioactive glass BG 45S5 with microsphere sizes between 75 and 125 μm. Formulation 2 was composed of BG 45S5 with microspheres of size 10 μm or less. Formulation 3 did not include any bioactive glass in its composition. The L/P ratios used for each formulation is displayed below in Table 1. Once the CPC was packed into the molds, the molds were placed in an oven at 37° C. The incubation times tested were 30 minutes, 1 hour, 4 hours, 15 hours, 24 hours, and 72 hours. After incubation, the CPC columns were allowed to cool for 5 minutes. After the cooling period the columns were removed and tested under compression at a test rate of 1 mm/min.

TABLE 1
The finalized liquid to powder ratios used

| Formulation | L/P Ratio |
|---|---|
| 1 | 0.3 |
| 2 | 0.3 |
| 3 | 0.35 |

Results

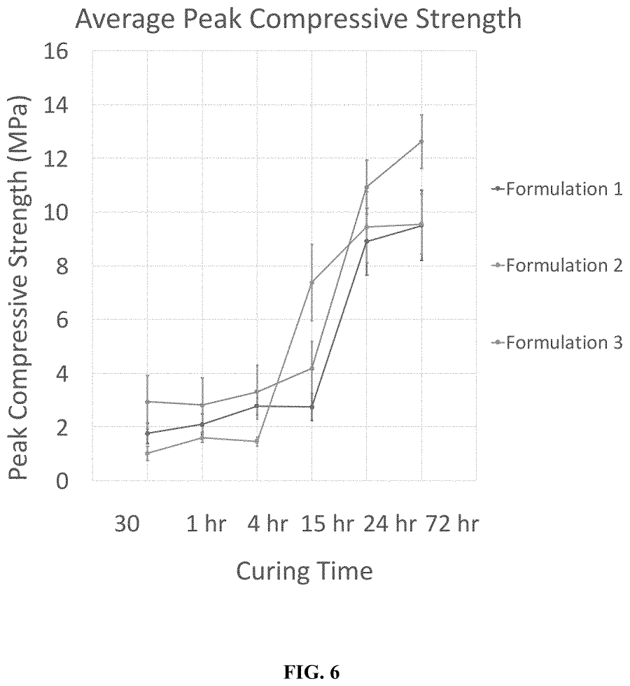

For all timepoints, as shown in the graph at FIG. 6, Formulation 3 consistently had the highest average peak compressive strength except for the 15 hour timepoint where Formulation 2 had the highest strength. For time points 30 min, 1 hour, 4 hour and 72 hour, Formulation 3 had a significantly higher peak compressive strength than Formulations 1 and 2 (p<0.05, n=4). It should also be noted that there was no significant difference in compressive strength between the 24 hour and 72 hour time points within each respective group. Overall, increasing the curing time led to an increase in the peak compressive strength for all the samples. However, the data reveals that while increases in cure time did increase the compressive strength, after 24 hours, there was not a significant difference in the strength of the pellets when compared to 72 hours.

Conclusions

Calcium phosphate cements can benefit from the addition of BG 45S5 due to its ability to promote the proliferation, differentiation, mineralization, and attachment of osteoblastic cells. Characterization of the compressive properties is an important step in determining if CPC-BG can become a viable bone graft substitute. We have demonstrated two CPC-BG formulations that are capable of providing compressive strength similar to native cancellous bone after 24 hours of curing.

It has further been observed in other studies that the smaller diameter bioactive glass particles (i.e., less than about 10 microns) showed greater interdigitation with the trabeculae of the cancellous bone. Accordingly, there is a desire to utilize a paste having relatively smaller bioactive glass particles in order to provide better interdigitation, without compromising compressive strength.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. A bone void filler for treating a bone defect, comprising:
a calcium phosphate material having therein a bioactive glass component, the bone void filler being osteostimulative, bioactive and flowable for injection through a syringe,
wherein the bioactive glass component comprises bioactive glass particles having an average diameter in the range of about 2 to 25 microns.

2. The bone void filler of claim 1, wherein the calcium phosphate material comprises 15 to 40% by wt. beta tricalcium phosphate.

3. The bone void filler of claim 1, further including 15 to 30% by wt. monocalcium phosphate monohydrate.

4. The bone void filler of claim 1, further including 5 to 15% by wt. hydroxyapatite.

5. The bone void filler of claim 1, further including 5 to 7% carboxy methyl cellulose.

6. The bone void filler of claim 1, further including 5% to 35% surfactant.

7. The bone void filler of claim 6, wherein the surfactant comprises copolymers of polypropylene polyethylene glycol.

8. The bone void filler of claim 1, wherein the bioactive glass component is in the range of 5 to 25% by wt. bioactive glass.

9. The bone void filler of claim 1, wherein the bioactive glass particles have a diameter less than about 10 microns.

10. The bone void filler of claim 1, wherein the bioactive glass component comprises 45S5 bioactive glass (45 wt % SiO₂, 24.5 wt % CaO, 24.5 wt % Na₂O and 6.0 wt % P₂O₅), boron bioactive glass (20 wt % CaO, 6 wt % Na₂O, 4 wt % P₂O₅, 51.6 wt % B₂O₃, 12 wt % K₂O, 5 wt % MgO, 0.4 wt % CuO, 1 wt % ZnO), or S53P4 (53 wt % SiO₂, 23 wt % Na₂O, 20 wt % CaO and 4 wt % P₂O₅).

11. The bone void filler of claim 1, wherein the filler has a minimum compressive strength of 1 mPa after hardening.

12. The bone void filler of claim 1, wherein the bone defect is a bone marrow lesion and the filler is configured for injection in a subchondral bone defect.

13. A kit for making a bone void filler for treating a bone defect, comprising:
(a) dry components of calcium phosphate and bioactive glass, and
(b) a liquid component comprising saline, citric acid, or sodium hydroxide solution;
wherein the dry material comprises 5 to 25% by wt. bioactive glass powder, the bioactive glass particles having an average diameter in the range of about 2 to 25 microns, 15 to 40% by wt. beta tricalcium phosphate powder, 15 to 30% by wt. monocalcium phosphate monohydrate, 5 to 15% by wt. hydroxyapatite, and 5 to 7% carboxy methyl cellulose; and
the liquid component comprises 0.5 M solution in a ratio of 2.2 gram dry material/cc of liquid.

14. The kit of claim 13, wherein the bioactive glass powder has an average diameter in the range of 50 to 200 microns.

15. The kit of claim 13, wherein the bioactive glass component comprises 45S5 bioactive glass (45 wt % SiO2, 24.5 wt % CaO, 24.5 wt % Na2O and 6.0 wt % P2O5), boron bioactive glass (20 wt % CaO, 6 wt % Na2O, 4 wt % P2O5, 51.6 wt % B2O3, 12 wt % K2O, 5 wt % MgO, 0.4 wt % CuO, 1 wt % ZnO), or S53P4 (53 wt % SiO2, 23 wt % Na2O, 20 wt % CaO and 4 wt % P2O5).

16. The kit of claim 13, further including 5% to 35% surfactant.

17. The kit of claim 16, wherein the surfactant comprises copolymers of polypropylene polyethylene glycol.

18. The kit of claim 13, further including a syringe delivery system.

19. The kit of claim 18, wherein the delivery system comprises a first syringe for containing the dry components, and a second syringe for containing the liquid components.

20. The kit of claim 18, wherein the second syringe is attachable to the first syringe through a connector.

21. The kit of claim 13, wherein the liquid to dry components ratio is in the range of about 0.25 to 0.4

22. The kit of claim 21, wherein the liquid to dry components ratio is in the range of about 0.3 to 0.35.
